(19) United States
(12) Patent Application Publication
(10) Pub. No.: US 2018/0284765 A1
(43) Pub. Date: Oct. 4, 2018

(54) EMERGENCY ACCESS TO AN INACTIVE VEHICLE

(71) Applicant: Faraday&Future Inc., Gardena, CA (US)

(72) Inventors: Michael Lambertus Hubertus Brouwer, Los Gatos, CA (US); Andrew Wayne Konecki, Pleasanton, CA (US)

(21) Appl. No.: 15/720,163

(22) Filed: Sep. 29, 2017

Related U.S. Application Data
(60) Provisional application No. 62/402,985, filed on Sep. 30, 2016.

(51) Int. Cl. G05D 1/00; B60R 25/102; B60R 25/25; B60R 25/24; A61B 5/1172; A61B 5/1171

(52) U.S. Cl. CPC G05D 1/0061; G05D 1/0088; B60R 25/102; B60R 25/25; H04W 4/90; A61B 5/1172; A61B 5/1176; A61B 5/1171; B60R 25/241

(57) ABSTRACT

In certain embodiments, a computer-implemented method of providing vehicle access can include receiving, from a user, a request to access a vehicle, determining whether the user is one of a set of approved users, determining a type of the request, and granting the user a limited-access to the vehicle when the user is not one of the set of approved users and the type of request is an emergency access request. The request can be received from a mobile computing device, such as a smart phone, laptop computer, wearable (e.g., smart watch), or other suitable computing device. In some cases, the request can further include an access code assigned to the user, an indication that an emergency button is activated on the mobile computing device, or an indication that an emergency 9-1-1 call has been made within a threshold period of time by the mobile computing device.

### CROSS-REFERENCE TO RELATED APPLICATIONS

This application claims the benefit of U.S. Provisional Application No. 62/402,985, filed Sep. 30, 2016, the entirety of which is hereby incorporated by reference.

### BACKGROUND

Approximately 240 million “9-1-1” calls are reported each year in the United States alone, which places an enormous burden on emergency response resources (e.g., police, fire, EMS, etc.) that can result in delays that could be life-threatening to a party in distress. Response times can vary depending on the circumstances of the emergency, notably with regard to the location of the site of the emergency and the relative distance of the emergency response resource.

Some types of emergencies have clear delineations of what the appropriate response should be and typically have the fastest response times. For instance, car accidents may involve the local police, EMS, and fire department, which can be deployed very quickly, depending on the location of the accident. However, some legitimate emergencies may not prompt an immediate response by emergency response services. For example, if a bicyclist crashed and was immobilized in a remote area, emergency services may place the response lower in priority than other more life-threatening emergencies, particularly when emergency resources are over-utilized and not immediately available. In another example, a person may feel unsafe in a particular area at night, but a 9-1-1 call may not receive the requisite attention from emergency services unless an actual emergency is occurring.

Some emergencies may not trigger any emergency services at all, despite the dire need of the distressed party. For example, if a person was admitted to the hospital after a serious accident, a close family member may lack personal transportation to get to the hospital, and transportation services (e.g., taxi, Uber®, Lyft®, etc.) may not be readily available. As such, emergency response services are not only limited in their coverage and response time, but also may only apply to a subset of the types of emergencies that a person may experience. Better solutions are needed to service a wider range of emergencies in a more timely manner.

### BRIEF SUMMARY

In certain embodiments, a computer-implemented method of providing vehicle access includes receiving, from a user, a request to access a vehicle, determining whether the user is one of a set of approved users, determining a type of the request, and granting the user a limited-access to the vehicle when the user is not one of the set of approved users and the type of request is an emergency access request. The request can be received from a mobile computing device, such as a smart phone, laptop computer, wearable (e.g., smart watch), or other suitable computing device. In some cases, the request can further include at least one of an access code assigned to the user, where the access code may be associated with gaining user access to user-non-approved vehicles under an emergency condition, an indication that an emergency button is activated on the mobile computing device, or an indication that an emergency 9-1-1 call has been made within a threshold period of time by the mobile computing device. The method can further include receiving data corresponding to the identity of the user from the mobile computing device (e.g., this can be associated with the access code or separately provided).

In some embodiments, the request can include biometrics data corresponding to the user, which can include at least one of fingerprint data, iris scan data, facial recognition data, deoxyribonucleic acid (DNA) data, or the like. The method may further include sending the biometrics data to an authentication server, and receiving confirmation that the biometrics data corresponds to the requesting user. In such cases, granting the user limited-access can be further based on receiving the confirmation that the biometrics data corresponds to the requesting user.

In further embodiments, determining the type of the request may include determining whether the request is at least one of a normal access request or an emergency access request. The limited-access to the vehicle may include initiating an advanced driver assistance system (ADAS) to automatically drive the user to one of a predetermined set of destinations. In some cases, the limited-access to the vehicle can include preventing manual control of the vehicle by the user. In some cases, the method can include contacting an owner of the vehicle, and informing the owner that the vehicle is in use by the user. Informing the owner that the vehicle is in use by the user can further include at least one of providing an identify of the user, or providing which of the predetermined set of destinations has been selected by the user. In some implementations, the method may further include receiving, from the user, a selection of one of the predetermined set of destinations, contacting a person or entity associated with the selected destination of the vehicle, and providing user data to the contacted person or entity, the user data corresponding to the emergency access request. The user data corresponding to the emergency access request may include at least one of image data of the user, the image data generated by one or more image sensors in the vehicle, or audio data of the user, the audio data generated by one or more microphones in the vehicle. The method may further include determining a location of the user, driving to the location of the user, and providing the limited-access to the vehicle to the user.

In certain embodiments, a system includes one or more processors, and one or more non-transitory computer-readable storage mediums containing instructions configured to cause the one or more processors to perform operations including receiving, from a user, a request to access a vehicle, determining whether the user is one of a set of approved users, determining a type of the request, and granting the user a limited-access to the vehicle when the user is not one of the set of approved users, and the type of request is an emergency access request. The request can further include at least one of an access code assigned to the user, the access code associated with gaining user access to user-non-approved vehicles under an emergency condition, an indication that an emergency button is activated on the mobile computing device, or an indication that an emergency “9-1-1” call has been made within a threshold period of time by the mobile computing device. In some cases, identification data that identifies the requesting user can be provided, which can be associated with the access code or separately provided. In some cases, the system can include additional instructions configured to cause the one or more processors to perform operations including contacting an owner of the vehicle, and informing the owner that the vehicle is in use by the user. In some embodiments, informing the owner that the vehicle is in use by the user further can include at least one of providing an identity of the user, or providing which of the predetermined set of destinations has been selected by the user.

In some embodiments, a computer-implemented method of providing vehicle access includes a means for receiving, from a user, a request to access a vehicle, a means for determining whether the user is one of a set of approved users, a means for determining a type of the request, and a means for granting the user a limited-access to the vehicle when the user is not one of the set of approved users, and the type of request is an emergency access request.

In certain embodiments, a computer-implemented method of providing vehicle access includes receiving, from a user, a request to access a vehicle, determining a type of the request, and granting the user access to the vehicle when the type of the request is an emergency access request.

### BRIEF DESCRIPTION OF THE DRAWINGS

The detailed description is set forth with reference to the accompanying figures.

FIG. 1 shows a user gaining emergency access to a vehicle, according to certain embodiments.

FIG. 2 shows a user utilizing limited-access capabilities of a vehicle after gaining emergency access, according to certain embodiments.

FIG. 3 shows the user receiving emergency response services from an informed receiving party, according to certain embodiments.

FIG. 4 shows a user requesting emergency access to a vehicle using a mobile computing device, according to certain embodiments.

FIG. 5 shows a number of biometric data types that can be used to authenticate a user, according to certain embodiments.

FIG. 6 shows an authentication request for biometric data from a user requesting emergency access to a vehicle, according to certain embodiments.

FIG. 7 shows a user accessing sensor resources in a vehicle while driving under emergency access conditions, according to certain embodiments.

FIG. 8 is a simplified flow chart showing a method of providing emergency access to a vehicle, according to certain embodiments.

FIG. 9 shows a system for providing emergency access to a vehicle, according to certain embodiments.

### DETAILED DESCRIPTION

Aspects of the present disclosure relate generally to vehicular systems, and in particular gaining emergency access to an inactive vehicle, according to certain embodiments.

In the following description, various embodiments of gaining emergency access to an inactive vehicle will be described. For purposes of explanation, specific configurations and details are set forth in order to provide a thorough understanding of the embodiments. However, it will be apparent to one skilled in the art that certain embodiments may be practiced without every disclosed detail. Furthermore, well-known features may be omitted or simplified in order not to obscure the embodiments described herein.

As previously mentioned, over-burdened emergency response resources across the U.S. have resulted in delays that can be life-threatening delays to parties in distress. As of 2016, there are over 250 million registered vehicles on the road in the United States, with many of them being utilized less than one hour per day. Unused vehicles can be found in virtually every populated area nationwide at any given time. In contrast, some estimates indicate about 50,000 emergency response vehicles (e.g., EMS vans, fire trucks, state/private sponsored vehicles, etc.) in the U.S., or roughly 1 emergency vehicle per 5,000 people. As such, unused vehicles are plentiful and readily available for use, while emergency response vehicles may be tens of minutes away or more, even in favorable conditions. In an emergency situation, having immediate access to transportation (e.g., a nearby unused vehicle) could save lives. Certain embodiments of the invention allow a person (a “user”) to gain emergency access to a vehicle that they otherwise would not be able to access.

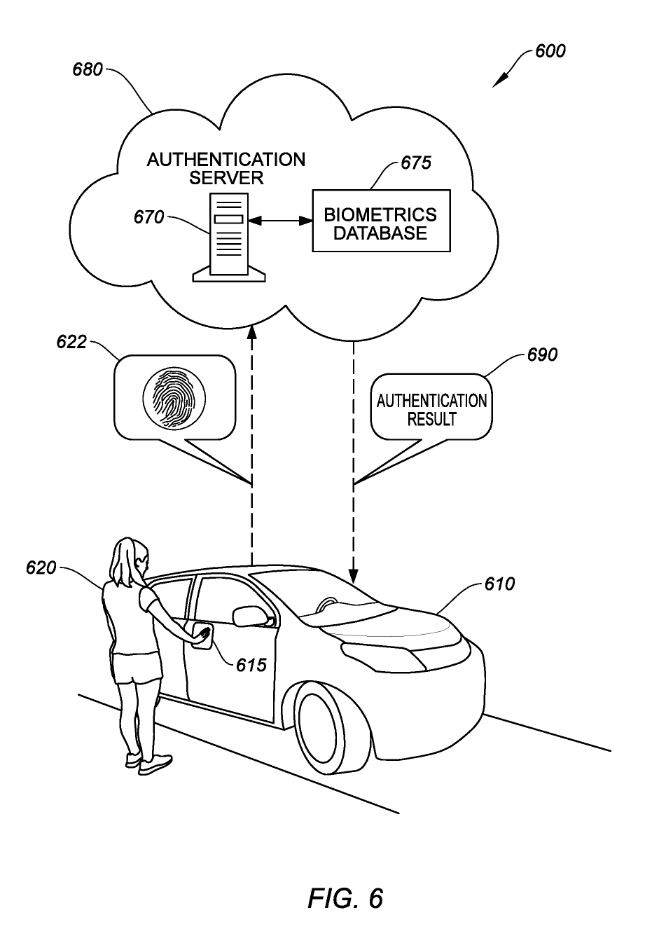

In some embodiments, a user can gain emergency access to a vehicle after an authentication process. Authentication can be performed with the use of a mobile computing device (e.g., in conjunction with a server) and corresponding software to implement the emergency access to the vehicle. The mobile computing device can be a smart phone, a wearable device (e.g., smart watch), a laptop computer, or the like. In some cases, biometric data can be used to authenticate the user. Some biometric data can include fingerprint data, iris scan data, facial recognition, DNA data, or other types of biometric data, and combinations thereof. The biometric data can be entered via the mobile computing device or directly on a vehicle (see, e.g., FIG. 6). In a fleet-type vehicle, a key fob may be used to authenticate the user. Once the authentication process is complete, the user may have limited access to a vehicle (e.g., doors unlock, limited driving capabilities, etc.), which may include a limited number of approved destinations (e.g., hospital, police station, etc.) and/or limited-access to vehicle resources (e.g., communications, sensor access, etc.), as further discussed below.

In certain embodiments, vehicle resources may be used to assist the user with the corresponding emergency. For instance, a vehicle may initiate audio resources to record the user describing the nature of the emergency, video resources to take a photo or video of the user (e.g., for identification purposes), and communication resources to contact a selected destination (e.g., hospital) to let them know the nature of the emergency, the condition of the user, and an estimated time of arrival, among other services. Additionally, the vehicle may contact the owner of the vehicle (e.g., via SMS, email, or other form of electronic communication) to let them know that their car is being used by an authenticated user for an emergency and provide user details such as the nature of the emergency, the selected destination, vehicle usage data (e.g., battery/fuel usage, resources used, wear/damage to vehicle during emergency use, etc.), an estimated time when emergency usage of the vehicle is expected to be complete, billing information (e.g., for use of the vehicle), and the like. The above-mentioned description is intended to provide a high-level overview of some of the novel features described herein. These concepts are discussed in further detail below in conjunction with the accompanying figures.

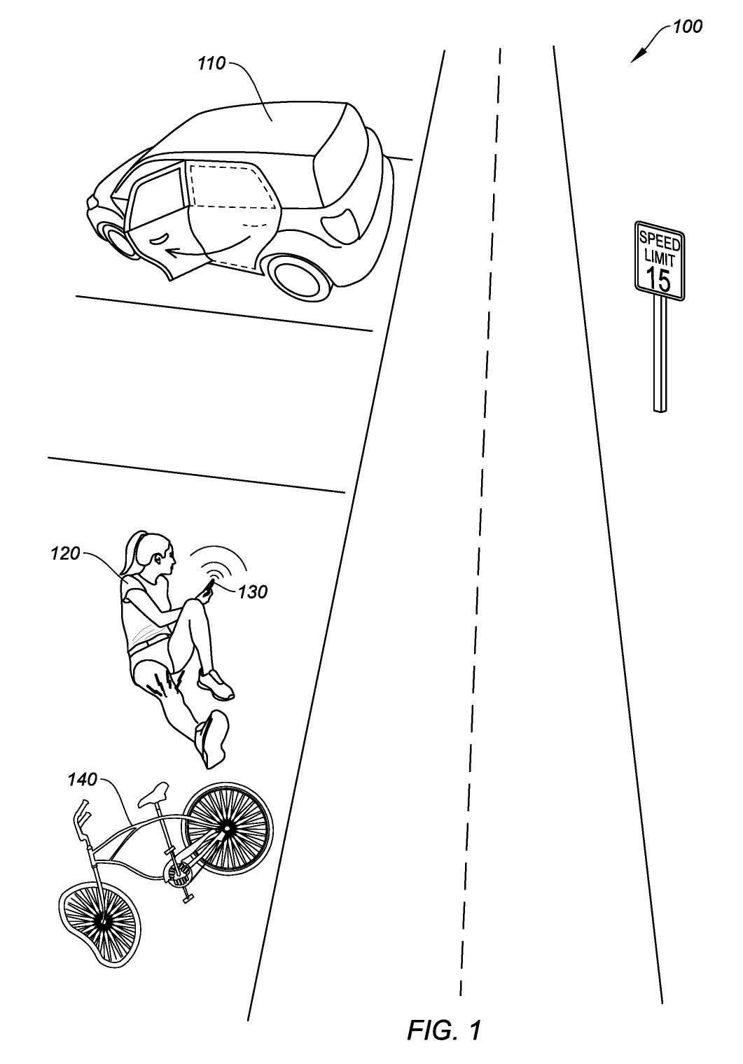

FIG. 1 shows user 120 gaining emergency access to a vehicle 110, according to certain embodiments. As mentioned above, in an emergency scenario, transportation may not be readily available. However, unused vehicles may be abundantly available and quickly accessible. Referring to FIG. 1, user 120 has crashed her bicycle 140, is immobile due to a broken leg, and requires immediate medical assistance. Noticing unused vehicle 110 nearby, user 120 accesses her mobile computing device 130 (e.g., smart phone) to request emergency vehicle access to vehicle 110. In response to the request, vehicle 110 grants emergency access to user 120 and opens a car door. In some cases, as shown in FIG. 1, the requesting user may not be physically capable of manually driving the vehicle on their own due to the extent of their injuries. In such cases, user 120 can perform a search for only those vehicles with ADAS capabilities, filter the results based on their relative distance from the requesting user and then send the emergency access request to one or more vehicles of the resultant list of ADAS-equipped vehicles. In this particular embodiment, user 120 can perform the filtering of vehicles and sending the request, however these tasks can be performed by a central server by automated or third-party controlled entities, as would be understood by one of ordinary skill in the art. In some embodiments, ADAS-equipped vehicles may be able to automatically drive to the location of user 120, thereby making the distance of vehicle 110 to user 120 less of a factor in providing immediate transportation to user 120 (e.g., if user 120 is immobile or severely movement restricted, autonomous vehicles that are 10 yards away versus 200 yards away may not significantly impact the overall transportation time to a location (e.g., hospital)).

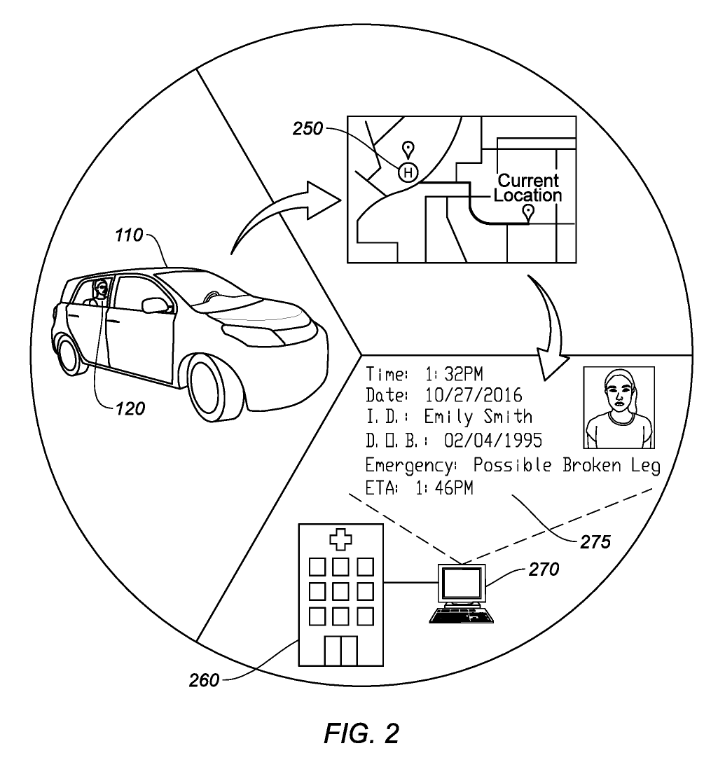

FIG. 2 shows user 120 having limited-access to vehicle 110 after gaining emergency access, according to certain embodiments. User 120 suffered a broken leg and is incapable of driving. User 120 enters the backseat of ADAS-equipped vehicle 110 and selects a desired destination 250. User 120 can select a desired destination using mobile computing device 130, providing voice commands to vehicle 110 (e.g., received by microphones disposed in the vehicle cabin), or the like.

In some embodiments, there may be a limited number of selectable destinations including one or more medical centers (e.g., hospitals, clinics), shelters, police stations, fire stations, schools, or the like. Alternatively or additionally, there may be vehicle usage limitations corresponding to a maximum amount of time user 120 may utilize vehicle 110, a maximum radial distance from a particular location (e.g., starting location), a maximum mileage, or any other suitable limitation. In some cases, travel may be limited to autonomous navigation (e.g., prevent manual control of vehicle 110). Referring back to FIG. 2, user 120 selects local hospital 260, and vehicle 110 begins driving to the selected destination.

During the drive to the selected destination, user 120 can indicate the nature of the emergency (e.g., verbally, through mobile computing device 130, etc.) and vehicle 110 can contact the intended destination (e.g., via web site, web portal, call to reception desk, etc.) with emergency related data. As shown in FIG. 2, vehicle 110 contacts hospital computer 270 and uploads various emergency data 275 including a time of the injury, a current date, identification data of user 120, user 120 date of birth, a nature of the emergency (e.g., possible broken leg), and estimated time of arrival (ETA). Different types of data can be sent including audio data (e.g., recording of user 120 providing details of the emergency using an audio sensor in vehicle 110), image data showing images/video of the broken leg (e.g., using image sensor(s) in vehicle 110), additional ETA data including a present location, speed, direction, etc., (e.g., global position system data, or similar navigational data) or any other type of data that would be relevant to the nature of the emergency. In some cases, the destination (e.g., hospital 260) may open a line of communication with user 120 (e.g., via telephone, SMS, email, video call, etc.) to get more information from user 120 and provide instructions, guidance, and the like. In some embodiments, gaining emergency access may automatically alert one or more pre-selected parties to the emergency (e.g., family, friends, etc.), which may have some access (e.g., set by user 120) to the details of the emergency (e.g., location, destination, sensor data (e.g., voice or image data), and the like).

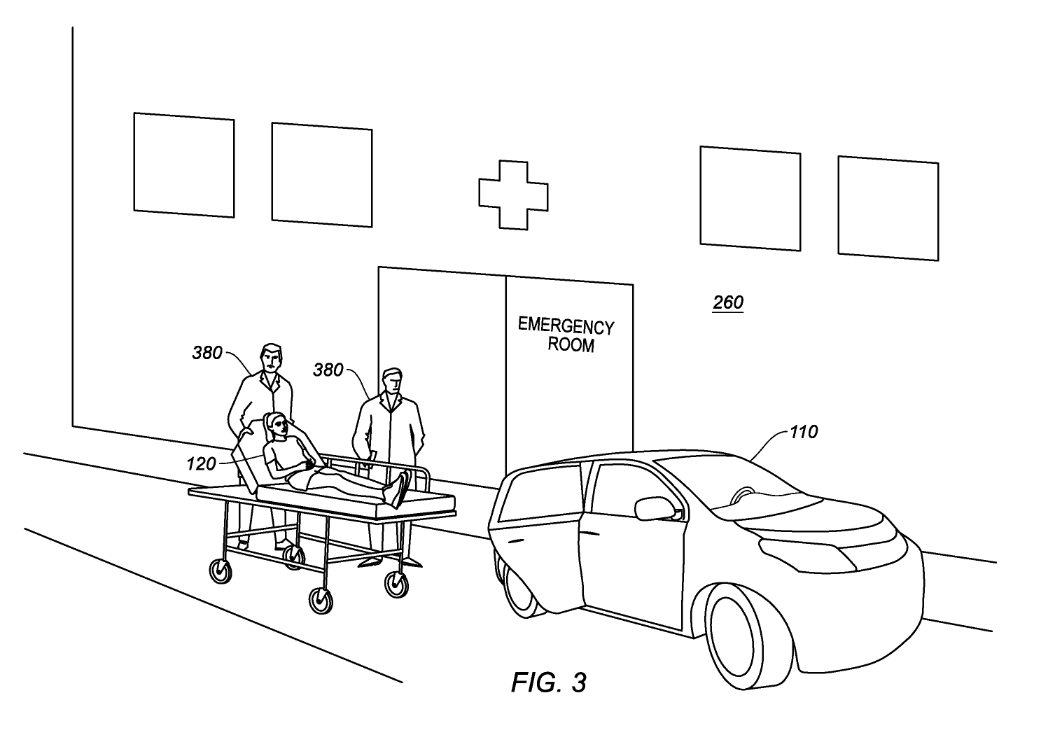

FIG. 3 shows user 120 receiving emergency response services from an informed receiving party, according to certain embodiments. The medical staff 380 of hospital 260 are informed after having received emergency data 275 and are ready at curbside to receive user 120. After user 120 no longer requires emergency access to vehicle 110, billing information can be sent to user 120 (e.g., vehicle usage fee, gas usage, damages incurred during use, etc.) and reported to the owner of vehicle 110. The owner can be notified via text, email, voice call, SMS, or other suitable form of communication that their vehicle 110 was used for an emergency service. In some embodiments, the owner of vehicle 110 can be immediately notified of when emergency access is granted, a present location (e.g., GPS data), the intended destination, an expected return time (e.g., in an ADAS-equipped vehicle 110), or other suitable information that an owner would want to know. Information pertaining to the nature of the emergency (e.g., emergency data 275) may be blocked from the owner per HIPAA rules and regulations. One of ordinary skill in the art would understand the many variations, modifications, and alternative embodiments thereof involving the handling of emergency data 275 and the information made available to the corresponding owner of vehicle 110.

The embodiments described in detail are limited to a few examples of typical emergency situations (e.g., bicycle crash, unsafe environment, etc.). It should be understood that the type of emergency situations that may be covered by the present invention can vary broadly in scope, and can be adaptive as new and previously unanticipated emergency situations emerge. Emergency conditions can include medical conditions (e.g., heart attack, sudden sickness, stroke, a fall, a victim of an assault, dog bite victim, of the like), escape conditions (e.g., user is being followed and wants to leave the area immediately), force of nature (e.g., escaping flood, fire, extreme weather conditions, etc.), or any other exigent condition. One of ordinary skill in the art would understand the many variations, modifications, and alternative embodiments thereof. Furthermore, the pool of vehicles that may be available for emergency access can be of any suitable size (e.g., nationwide, statewide, county wide) or type (e.g., fleet vehicles, cars, trucks, motorcycles, etc.).

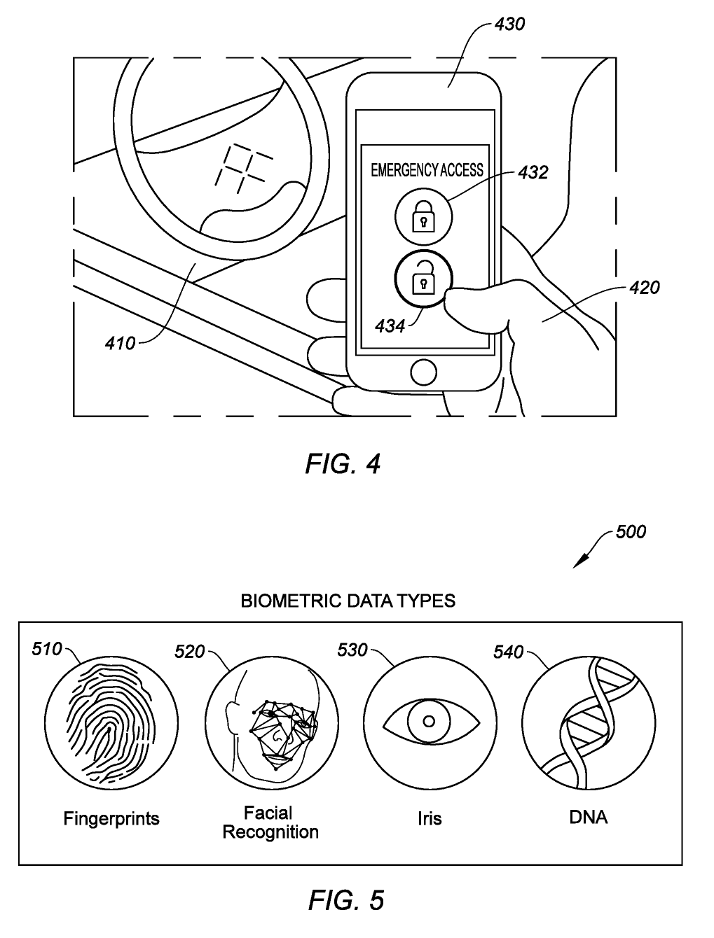

FIG. 4 shows user 420 requesting emergency access to vehicle 410 using mobile computing device 430, according to certain embodiments. Mobile computing device 430 can be a smart phone, wearable device (e.g., smart watch, smart glasses, etc.), laptop computer, or the like. In some cases, mobile computing device 430 may be a non-conventional mobile communication devices including another vehicle. For instance, if a large number of people need emergency transportation, a user can request emergency access to additional cars from their emergency-accessed vehicle (e.g., a vehicle having communication capabilities can be used to request emergency access to an additional vehicle).

In some embodiments, user 420 can utilize an emergency-access application on mobile computing device 430 to request emergency access. For instance, button 434 may request emergency access and button 432 may end the emergency access (e.g., when user 420 reaches their destination). The emergency access request can be made from user-to-vehicle, user-to-cloud, user-to-other entity, user-to-cloud-to-vehicle, etc., as further discussed below with respect to FIG. 6. Any suitable form of electronic communication can be used including Bluetooth® variants, infra-red, RF, ZigBee, Wi-Fi, cellular, and the like.

In some embodiments, user 420 may be preregistered and/or prescreened. For example, user 420 may have previously entered information that can be used for authentication purposes. For instance, user 420 may have an account that contains user information data (e.g., identity, account status, usage statistics, account balances from use, etc.), user identification data (e.g., biometrics data), or other information that can be used to verify that user 420 is who they claim to be. That way, user 420 can be authenticated at the time an emergency access request is made.

In certain embodiments, emergency access may be contingent upon an expected usage of the vehicle. For example, if an owner is expecting to use their vehicle very soon (e.g., in 15 minutes), then other immediately accessible vehicles may be provided. In some cases, expected owner usage may not be a factor in limiting emergency access. In cases where fleet vehicles are being used (e.g., cars not owned by a particular user), if a first user was scheduled to use a particular vehicle at a given time, and that vehicle becomes unavailable because of an emergency use case for a second user, another fleet vehicle may be deployed to replace the vehicle for the first user.

Referring back to FIG. 4, user 420 can use mobile computing device 430 to provide additional information such as the emergency type, desired destination (e.g., could have limited selection), expected time of use, etc. In some cases, where user 420 becomes incapacitated (e.g., user 420 cannot speak, type, is unconscious), certain default steps may be performed including destination selection (e.g., medical center), auto-communication (e.g., with medical center, police), auto-sensor usage (e.g., image, video, audio of user 420), auto-sending emergency data, etc., and the like. One of ordinary skill in the art would understand the many variations, modifications, and alternative embodiments thereof.

FIG. 4 shows the use of a smart phone (e.g., button 434) to request emergency access, however other triggers for emergency access to vehicle 410 may be used. Alternatively or additionally, triggers can include calls made to emergency services (e.g., 9-1-1), entering a special code uniquely assigned to user 420, a signal originating from an approved third party (e.g., OnStar®, police services, etc.), an indication that a child or pet is in danger (e.g., child or pet locked in hot vehicle), an email, SMS, or other message with some authentication measures (discussed below), key fobs, biometric devices (discussed below), or the like.

FIG. 5 shows a number of biometric data types 500 that can be used to authenticate a user, according to certain embodiments. Biometrics can be metrics that relate to human characteristics and may be used for authentication purposes (e.g., verifying the identity of a person). Biometric identifiers may be distinctive, measurable characteristics used to label and describe individuals (users), and are typically categorized as physiological or behavioral characteristics. Physiological characteristics can be related to the shape of the body. Examples include, but are not limited to fingerprint data 510, face recognition data 520, iris data 530, and DNA data 540, to name a few. Fingerprint data 510 may include a thumb print, one or more other digits, or a combination thereof. Fingerprint data 510 can be detected on mobile computing device 430, on a touch sensitive portion of vehicle 410, or other suitable biometric device. Face recognition data 520 and iris data 530 can be collected by one or more image sensors on mobile computing device 430 (e.g., video camera), on vehicle 410, or other device. DNA data 540 can be collected from blood, hair, finger nails, mouth swabs, blood stains, saliva, straws, and any number of other sources that has had contact with the body, and may be analyzed as would be understood by one of ordinary skill in the art.

Some other less commonly used physiological biometric data can include audio data (e.g., voice recognition), palm print, hand geometry, retina data, odor/scent data, palm veins, and more. Behavioral characteristics are typically related to the pattern of behavior of a person (user), including but not limited to a typing rhythm, walking gait, voice characteristics, etc. Any combination of biometrics may be used.

More traditional means of access control include token-based identification systems, such as a driver's license or passport, and knowledge-based identification systems, such as a password or personal identification number. Since biometric identifiers are unique to individuals, they may be more reliable in verifying identity than token and knowledge-based methods. However, there may be privacy concerns, which falls outside the purview of the present disclosure. In some embodiments, both biometric identification and token-based identification (e.g., mobile computing device 430) can be used. Any combination of identifying and authentication technologies may be used in any suitable manner, as would be understood by one of ordinary skill in the art.

FIG. 6 shows an authentication request using biometric data 622 from user 620 to gain emergency access to vehicle 610, according to certain embodiments. User 620 is shown at location 600 where she feels unsafe, believes that she is being followed, and requires immediate access to vehicle 610 to get to a safer location (e.g., police department). User 620 can provide biometric data 622 (e.g., fingerprint data) to touch sensitive surface 615 on vehicle 610. Vehicle 610 can send biometric data 622 to authentication server 670 residing the cloud 680. Authentication server 670 may then compare biometric data 622 with corresponding biometrics data in biometrics database 675 to see if there is a match. Authentication result 690 is sent back to vehicle 610 and emergency access can be granted (e.g., a biometrics match) or denied (e.g., no match found) based on the result. The authentication process can be performed very quickly (e.g., less than a few seconds) to accommodate exigent circumstances.

In some embodiments, biometrics database 675 can exist in the cloud (e.g., cloud 680), in vehicle 610, or in other suitable locations. However, given the sensitivity of value of a person's biometric data, biometrics database 675 would likely be accessible via authentication server 670 (and possibly government regulated) and likely not to individual users. Biometrics database 675 may include biometrics data that user 620 previously sent when pre-registering, as discussed above. In some embodiments, biometric data 622 may be encrypted for improved security (e.g., via mobile computing device 430). In such cases, vehicle 610 may receive and send biometric data 622 to authentication server 670 without decrypting the data, leaving decryption processes to be performed in the cloud. Similarly, decoding processes (e.g., analyzing biometric data) can done in the cloud. In some implementations, vehicle 610 does not save any received biometric data 622 and deletes any memory of biometric data 622 after it is sent to authentication server 670 for security purposes, as would be understood by one of ordinary skill in the art. Authentication result 690 can include an indication of whether biometric data 622 matched corresponding biometrics data for user 620 in biometrics database 675.

Communication between vehicle 610 and authentication server 670 can be performed using any suitable communications protocol including, but not limited to cellular, RF, Wi-Fi, or the like. If authentication result 690 is positive (e.g., a positive match), then user 620 may gain emergency access (e.g., limited-access) to vehicle 610, as further discussed above. If authentication result 690 is negative, some embodiments may allow one or more additional attempts using biometrics or other approved authentication methodologies. In some implementations, a denial of access may allow user 620 to enter vehicle 610 and lock the doors for safety. In some cases, certain access to media, telecommunications (to contact the authorities, and/or sensors (image sensors—to take a photo of an assailant outside vehicle 610) may be allowable. In some instances where an emergency situation is detected despite a negative authentication, emergency may be granted depending on the circumstances. For example, if user 620 was not authenticated, but was granted access to the cabin of vehicle 610 (but no driving capabilities) and an assailant began trying to break into the vehicle (e.g., detected by image/audio sensors in vehicle 610), then immediate emergency access may be granted (e.g., auto-drive to closest police station and send relevant emergency data). One of ordinary skill in the art would understand the many variations, modifications, and alternative embodiments thereof.

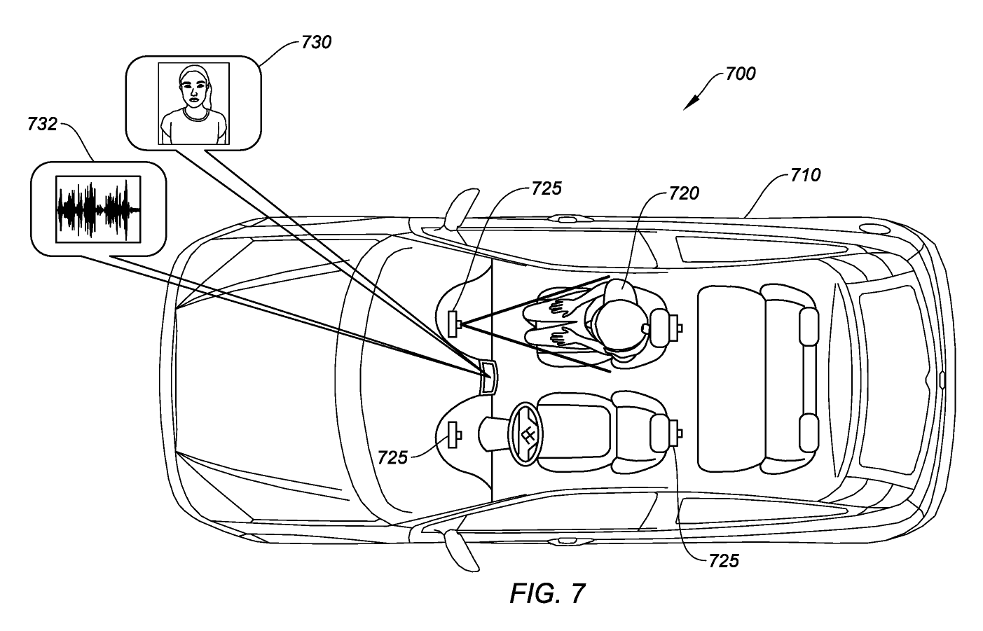

FIG. 7 shows user 720 accessing sensor resources in vehicle 710 while driving under emergency access conditions, according to certain embodiments. Vehicle 710 includes sensors 725, which may include image sensors (e.g., video camera), audio sensors (e.g., microphone), or the like.

As described above, vehicle 710 can provide services during transit such as access to certain sensors, communication, and media resources. For instance, if user 720 needed emergency access to vehicle 710 because she was being attacked, she can contact the police, and provide audio and video information about herself and the attacker (e.g., if available). Vehicle can automatically send this information if user 720 is incapacitated or unable to speak. For example, vehicle 710 can relay important information about the scene of the attack including a description of the attacker (e.g., video data 730 and/or audio data 732), the location of the attack, other people that were present at the scene (e.g., via image data), etc.

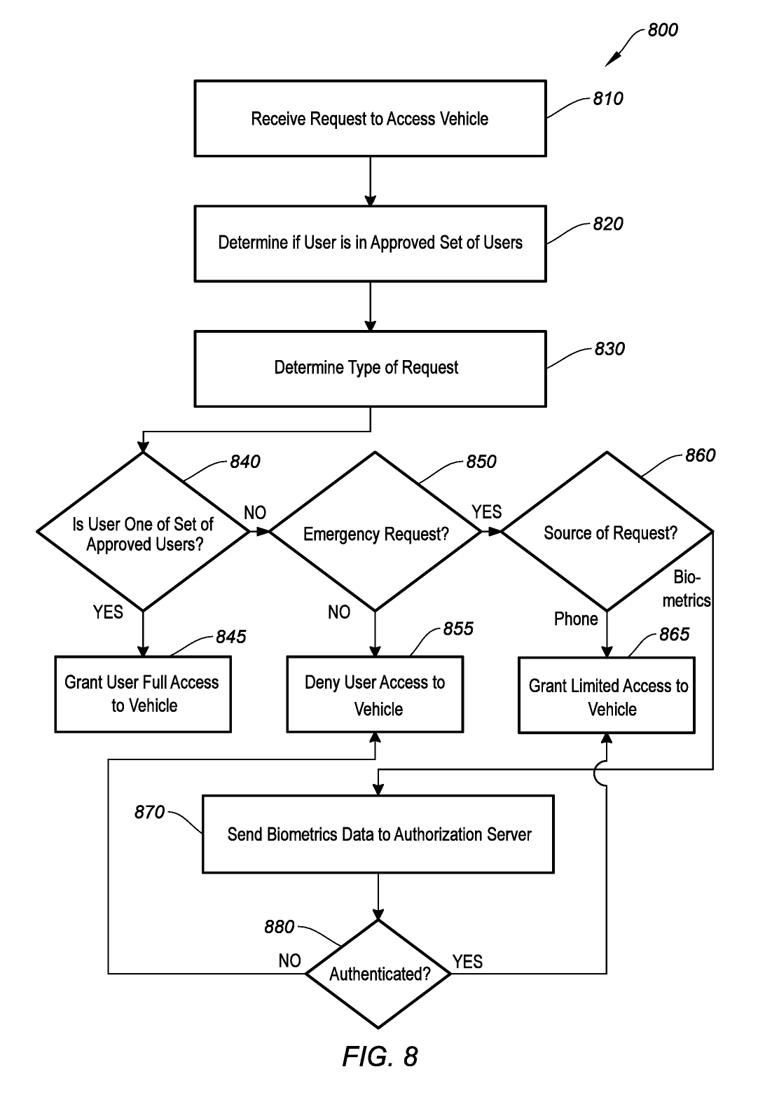

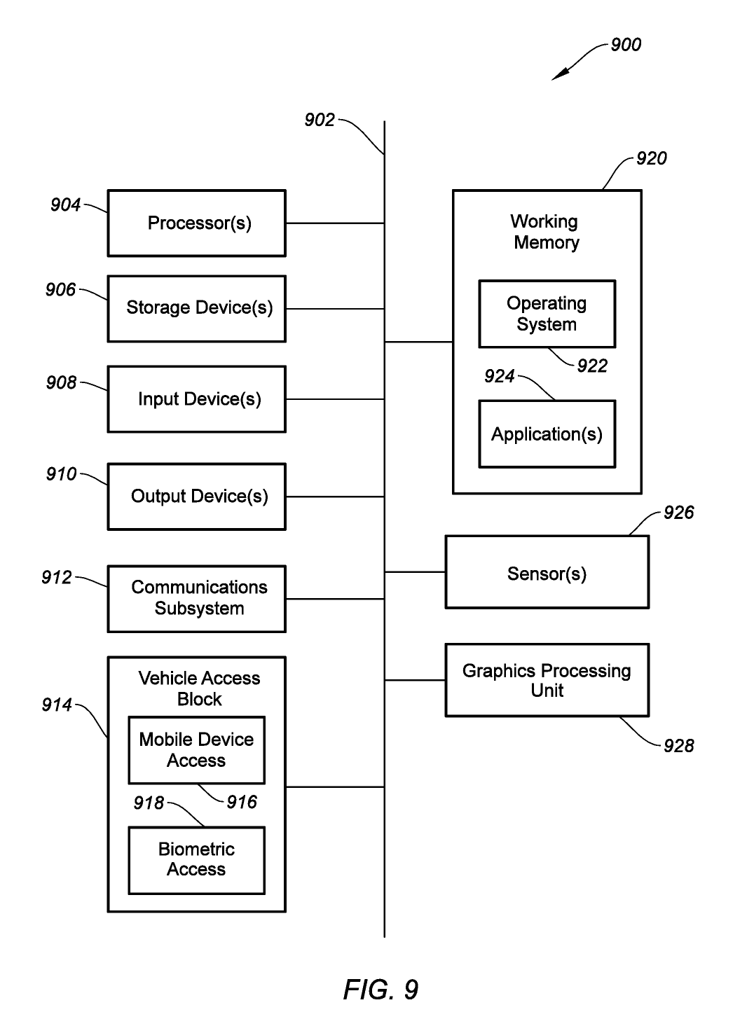

FIG. 8 is a simplified flow chart showing a method 800 of providing emergency access to a vehicle, according to certain embodiments. Method 800 can be performed by processing logic that may comprise hardware (circuitry, dedicated logic, etc.), software operating on appropriate hardware (such as a general purpose computing system or a dedicated machine), firmware (embedded software), or any combination thereof. In certain embodiments, method 800 can be performed by processor(s) 904 of FIG. 9, or other suitable computing device.

At step 810, method 800 can include receiving, from a user (120), a request to access a vehicle (110); according to certain embodiments. The request can come a mobile input device (e.g., smart phone, wearable (e.g., smart watch, smart glasses), laptop computer, desktop computer, from computing devices in other vehicles or locations, from other approved entities (e.g., police, hospital, OnStar®-type services, etc.), from one or more servers on the cloud or other remote location, and the like. The user may be preregistered so that the user can be quickly identifiable, billable, etc. Alternatively or additionally, biometrics can be used to identify the user, as further discussed above. The request can be received via any suitable communications protocol including, but not limited to, Bluetooth® variants, infra-red, RF, ZigBee, Wi-Fi, cellular variants, and the like.

In some embodiments, a different party can make a request for the user. For example, if a user is injured but does not have emergency request capabilities (e.g., no smart phone available), a second person can request access to the vehicle for the user and may assume the responsibilities associated with the service (e.g., costs, liability for damage to the vehicle, possible liability for safety of the user, etc.).

At step 820, method 800 can include determining whether the user is one of a set of approved users, according to certain embodiments. An approved user may be one or more owners of the vehicle, or people that the owner has identified as approved users (e.g., relatives, friends, etc.) via a user account or other suitable means as would be understood by one of ordinary skill in the art. A user that is not approved can be anyone that has not been expressly approved by the owner (e.g., in their user account).

At step 830, method 800 can include determining a type of the request, according to certain embodiments. The type of request can include an emergency access request or a non-emergency access request. An emergency access request can be a request to gain immediate access to a vehicle due to exigent circumstances. Emergency conditions can include medical conditions (e.g., injury, heart attack, sudden sickness, stroke, a fall, a victim of an assault, dog bite victim, of the like), escape conditions (e.g., user is being followed and wants to leave the area immediately), force of nature (e.g., escaping flood, fire, extreme weather conditions, etc.), or any other situation that may make you want to call 9-1-1 and/or escape a situation. One of ordinary skill in the art would understand the many variations, modifications, and alternative embodiments thereof. Emergency access requests are typically made by users that are not previously approved.

A non-emergency request can be a request for user access to the vehicle for normal everyday use. Non-emergency requests are typically made by those that are of a set of approved users of the vehicle.

At step 840, method 800 includes determining whether user 120 is one of set of approved users. When user 120 is of a set of approved users (e.g., an owner of vehicle 110), then user 120 can be granted full access to vehicle with no restrictions (step 845). When user 120 is not one of a set of approved users, then method 800 continues on to step 850.

At step 850, method 800 can include determining whether the request is an emergency access request. If the request is not an emergency access request (e.g., a non-emergency request), then user 120 can be denied user access to vehicle 110 (step 855). If the type of request is an emergency access request, then method 800 continues to step 860.

At step 860, method 800 can include determining the source of the request. If the request is received from a mobile input device (130), then user 120 can be granted limited-access to vehicle 110, as described above with respect to FIGS. 1-3. In some embodiments, the request can further include at least one of an access code assigned to the user, where the access code associated with gaining access to non-approved vehicles under an emergency condition, an indication that an emergency button (e.g., button 434) is activated on the mobile computing device (see, e.g., FIG. 4) or an indication that an emergency 9-1-1 call has been made within a threshold period of time by the mobile computing device. For example, if the user has called 9-1-1 in the last 30 minutes, or other suitable period of time (e.g., last 5 minutes, last hour, etc.). The decision to grant limited-access to the vehicle may further be based on these additional factors (e.g., emergency button, etc.) or other factors, One of ordinary skill in the art would understand the many variations, modifications, and alternative embodiments thereof.

If the source of the request includes biometric data, then vehicle 110 can send the biometrics data to an authentication server to authenticate the user, as described above with respect to FIG. 6. The biometrics data can include at least one of fingerprint data, iris scan data, facial recognition data, DNA data, or the like, as discussed above with respect to FIG. 5.

At step 880, method 800 includes granting limited access to the vehicle (step 865) if the confirmation is received from the authentication server that the biometrics data corresponds to the requesting user, or denying user access to the vehicle (step 855) if the authentication server returns a result indicating that the biometrics data does not correspond to the requesting user.

In some embodiments, granting limited-access to the vehicle can include initiating an ADAS to automatically drive the user to one of a predetermined set of destinations, preventing manual control of the vehicle by the user, contacting an owner of the vehicle and informing the owner that the vehicle is in use by the user, including the identity of the user and the destination selected by the user (or any suitable information, as discussed above with respect to FIG. 3. In some cases, when a user is granted limited-access to the vehicle, the vehicle may receive a selection of one of a predetermined set of destinations, contact the selected predetermined destination (e.g., a receptionist), and provide user data corresponding to circumstances of the user emergency access request. The circumstances of the user emergency access request can include image data of the user, the image data generated by one or more image sensors in the vehicle, audio data of the user, the audio data generated by one or more microphones in the vehicle, or other data from any vehicle resource, as would be understood by one of ordinary skill in the art. In some ADAS-equipped vehicles, once limited-access is granted, the vehicle (e.g., processor(s) 904) may determine a location of the user, drive to the location of the user, and provide the limited-access to the user upon arrival.

It should be appreciated that the specific steps illustrated in FIG. 8 provide a particular method 800 of providing emergency access to a vehicle, according to certain embodiments. Other sequences of steps may also be performed according to alternative embodiments. For example, alternative embodiments may perform the steps outlined above in a different order. Moreover, the individual steps illustrated in FIG. 8 may include multiple sub-steps that may be performed in various sequences as appropriate to the individual step. Furthermore, additional steps may be added or removed depending on the particular applications. It should be noted that certain figures are referred to as references to specific entities (e.g., user 120, vehicle 110, etc.) in method 800. It should be understood that any of the concepts discussed throughout this disclosure (e.g., FIGS. 1-7 and 9) can be applied to method 800 or variants thereof. One of ordinary skill in the art would recognize and appreciate many variations, modifications, and alternatives of method 800.

FIG. 9 shows a computer system 900 for providing emergency access to a vehicle, according to certain embodiments. Computer system 900 can be used to implement and/or control any of the computer systems/devices (e.g., sensors, communication systems, access grant/denial, etc.) described above with respect to FIGS. 1-8. As shown in FIG. 9, computer system 900 can include one or more processor(s) 904 to communicate with a number of peripheral devices via a bus subsystem 902. These peripheral devices can include storage devices 906 (including long term storage), user input device(s) 908 (e.g. image-based sensors), user output device(s) 910 (e.g., video display to alert user to POI), communications subsystem 912, vehicle access block 914, graphics processing unit 928, sensors 926, and working memory 920. System blocks may be excluded or some may be added, as would be understood by one of ordinary skill in the art.

In certain embodiments, processor(s) 904 may include one or more microprocessors (μCs) and may control the operation of computer system 900. Alternatively, processor(s) 904 may include one or more microcontrollers (MCUs), digital signal processors (DSPs), or the like, with supporting hardware and/or firmware (e.g., memory, programmable I/Os, etc.), as would be appreciated by one of ordinary skill in the art. In some embodiments, processor(s) 904 may be configured to control aspects of emergency access requests (see, e.g., FIG. 1), receiving, processing, and sending emergency data (see, e.g., FIG. 2), authentication requests (see, e.g., FIG. 6), and other processes described herein, as would be understood by one of ordinary skill in the art.

In some embodiments, a graphics processing unit (GPU) 928 can operate independently or in conjunction with processor(s) 904 to control one or more output devices 910. For example, output devices 910 may include one or more displays in a vehicle. GPU 928 and/or processor(s) 904 may control graphics, user interface (UI) characteristics, or other display-based functions, as would be appreciated by one of ordinary skill in the art. For instance, GPU 928 may control a UI for user 120 to enter emergency information as discussed above with respect to FIG. 2.

In some examples, internal bus subsystem 902 can provide a mechanism for letting the various components and subsystems of computer system 900 communicate with each other as intended. Although internal bus subsystem 902 is shown schematically as a single bus, alternative embodiments of the bus subsystem can utilize multiple buses. Additionally, communications subsystem 912 can serve as an interface for communicating data between computer system 900 and other computer systems or networks (e.g., in the cloud). Embodiments of communications subsystem 912 can include wired interfaces (e.g., Ethernet, CAN, RS232, RS485, etc.) or wireless interfaces (e.g., ZigBee, Wi-Fi, cellular, etc.).

In some cases, input device(s) 908 can include one or more microphones (e.g., to receive emergency data from user 120), keyboard (e.g., to physically enter emergency data), pointing devices (e.g., mouse, trackball, touchpad, etc.—to select a predetermined destination, navigate dropdown menus for providing emergency data, etc.), a barcode scanner, a touch-screen incorporated into a display, audio input devices (e.g., voice recognition systems, etc.), Human Machine Interfaces (HMI) and other types of input devices. In general, use of the term “input device” is intended to include all possible types of devices and mechanisms for inputting information into computer system 900. Additionally, user interface output devices 910 can include a display subsystem or non-visual displays such as audio output devices, etc. The display subsystem can be any known type of display device. In general, use of the term “output device” is intended to include all possible types of devices and mechanisms for outputting information from computer system 900.

Storage devices 906 can include memory subsystems and file/disk storage subsystems (not shown), which can be non-transitory computer-readable storage media that can store program code and/or data that provide the functionality of embodiments of the present disclosure (e.g., method 800). In some embodiments, storage devices 906 can include a number of memories including main random access memory (RAM) for storage of instructions and data during program execution and read-only memory (ROM) in which fixed instructions may be stored. Storage devices 906 can provide persistent (i.e., non-volatile) storage for program and data files, and can include a magnetic or solid-state hard disk drive, an optical drive along with associated removable media (e.g., CD-ROM, DVD, Blu-Ray, etc.), a removable flash memory-based drive or card, and/or other types of storage media known in the art.

Computer system 900 can also include software elements, shown as being currently located within working memory 920, including an operating system 922, device drivers, executable libraries, and/or other code, such as one or more application programs 924, which may comprise computer programs provided by various implementations, and/or may be designed to implement methods, and/or configure systems, provided by other implementations, as described herein. Merely by way of example, one or more procedures described with respect to method 800 discussed above can be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a computer-readable storage medium, such as the storage device(s) 906 described above. In some cases, the storage medium might be incorporated within a computer system, such as computer system 900. In other implementations, the storage medium might be separate from a computer system (e.g., a removable medium, such as a compact disc), and/or provided in an installation package, such that the storage medium can be used to program, configure and/or adapt a general purpose computer with the instructions/code stored thereon. These instructions might take the form of executable code, which may be executable by computer system 900 and/or might take the form of source and/or installable code, which, upon compilation and/or installation on computer system 900 (e.g., using any of a variety of generally available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed. In some implementations, one or more elements of computer system 900 may be omitted or may be implemented separate from the illustrated system. For example, processor(s) 904 and/or other elements may be implemented separate from input device(s) 908. In one implementation, the processor may be configured to receive images from one or more image-based sensors (e.g., video cameras).

Some implementations may employ a computer system (such as computer system 900) to perform methods in accordance with the disclosure. For example, some or all of the procedures of the described methods (e.g., methods 400-600) may be performed by computer system 900 in response to processor(s) 904 executing one or more sequences of one or more instructions (which might be incorporated into operating system 922 and/or other code, such as an application program 924) contained in the working memory 920. Such instructions may be read into working memory 920 from another computer-readable medium, such as one or more of storage device(s) 906. Merely by way of example, execution of the sequences of instructions contained in working memory 920 might cause processor(s) 904 to perform one or more procedures of the methods described herein.

The terms “machine-readable medium” and “computer-readable medium,” as used herein, refer to any medium that participates in providing data that causes a machine to operate in a specific fashion. In some implementations implemented using computer system 900, various computer-readable media might be involved in providing instructions/code to processor(s) 904 for execution and/or might be used to store and/or carry such instructions/code (e.g., as signals). In many implementations, a computer-readable medium may be a physical and/or tangible storage medium. Such a medium may take many forms, including but not limited to, non-volatile media, volatile media, and transmission media. Non-volatile media include, for example, optical and/or magnetic disks, such as the storage device(s) 906. Volatile media include, without limitation, dynamic memory, such as working memory 920. Transmission media include, without limitation, coaxial cables, copper wire, and fiber optics, including the wires that comprise bus subsystem 902, as well as the various components of communications subsystem 912 (and/or the media by which communications subsystem 912 provides communication with other devices). Hence, transmission media can also take the form of waves (including without limitation radio, acoustic and/or light waves, such as those generated during radio-wave and infrared data communications).

Common forms of physical and/or tangible computer-readable media include, for example, a floppy disk, a flexible disk, hard disk, magnetic tape, or any other magnetic medium, a CD-ROM, any other optical medium, a RAM, a PROM, EPROM, a FLASH-EPROM, any other memory chip or cartridge, a carrier wave as described hereinafter, or any other medium from which a computer can read instructions and/or code.

Various forms of computer-readable media may be involved in carrying one or more sequences of one or more instructions to processor(s) 904 for execution. Merely by way of example, the instructions may initially be carried on a magnetic disk and/or optical disc of a remote computer. A remote computer might load the instructions into its dynamic memory and send the instructions as signals over a transmission medium to be received and/or executed by computer system 900. These signals, which might be in the form of electromagnetic signals, acoustic signals, optical signals and/or the like, are all examples of carrier waves on which instructions can be encoded, in accordance with various implementations of the invention.

Computer system 900 might also include a communications subsystem 912, which can include without limitation, a modem, a network card (wireless or wired), an infrared communication device, a wireless communication device and/or chipset (such as a Bluetooth device, an 802.11 device, a Wi-Fi device, a WiMAX device, cellular communication facilities, etc.), and/or the like. Communications subsystem 912 may permit data to be exchanged with a network, other computer systems, and/or any other devices described herein. In many implementations, computer system 900 can further comprise a non-transitory working memory 920, which can include a RAM or ROM device, as described above.

In some embodiments, sensor(s) 926 can include any type of image based sensor or video system including, but not limited to, digital camera systems, IR sensors, LIDAR systems, audio-based systems (e.g., ultrasonic, sonar, etc.), or the like. For example, sensors(s) 926 can include the image-based sensors discussed above with respect to FIGS. 2 and 7. Sensor(s) 926 can further include audio sensors (e.g., microphones), touch-based sensors (e.g., touch sensitive surface 615), or the like. Sensor(s) 926 can be subsumed (be a part of) input device(s) 908 and output devices (910). Thus, sensor(s) 926 and/or input device(s) 908/output device(s) 910 can include and or control all aspects of input, output, sensing, and the like, as described above with respect to FIGS. 1-8.

Vehicle access block 914 can include mobile device access block 916 and biometric access block 918, and may control aspects of determining whether to grant vehicle access to a user, the type of access granted (e.g., full/limited access), determining the type of request (e.g., emergency request), the type of user (e.g., owner, non-owner), and the like, as further discussed above. Mobile device access block 916 may control aspects of receiving access requests from mobile electronic devices, determining the type of request being received, and the like, as discussed above with respect to FIG. 4. Biometric access block 918 can perform functions associated with granting access using user biometric data. Biometric access block 918 can receive biometric data, store biometric data (permanently or temporarily), control the sending authentication requests using biometric data via communications subsystem 912, as discussed above with respect to FIGS. 5 and 6. Aspects of some or all of vehicle access block 914 may be subsumed by other system blocks in computer system 900 (e.g., processor(s) 904 and working memory 920). In some embodiments, some or all of the functions associated with vehicle access block 914 may be performed offsite (i.e., not by the vehicle). For instance, decisions to grant emergency access may be made in a server in the cloud and the vehicle can merely either grant or deny access based on the determination made by a separate entity. One of ordinary skill in the art would understand the many variations, modifications, and alternative embodiments thereof.

It should be appreciated that computer system 900 is illustrative and not intended to limit embodiments of the present disclosure. Many other configurations having more or fewer components than computer system 900 are possible. Further, computer system 900 may be combined with, subsumed by in part or in whole, or otherwise used in conjunction with system 900, as would be understood by one of ordinary skill in the art with the benefit of this disclosure.

Most embodiments utilize at least one network that would be familiar to those skilled in the art for supporting communications using any of a variety of commercially available protocols, such as TCP/IP, UDP, OSI, FTP, UPnP, NFS, CIFS, and the like. The network can be, for example, a local area network, a wide-area network, a virtual private network, the Internet, an intranet, an extranet, a public switched telephone network, an infrared network, a wireless network, and any combination thereof. Non-transitory storage media and computer-readable storage media for containing code, or portions of code, can include any appropriate media known or used in the art such as, but not limited to, volatile and non-volatile, removable and non-removable media implemented in any method or technology for storage of information such as computer-readable instructions, data structures, program modules or other data, including RAM, ROM, Electrically Erasable Programmable Read-Only Memory (EEPROM), flash memory or other memory technology, CD-ROM, DVD or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments. However, computer-readable storage media does not include transitory media such as carrier waves or the like.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. The phrase “based on” should be understood to be open-ended, and not limiting in any way, and is intended to be interpreted or otherwise read as “based at least in part on,” where appropriate. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

What is claimed is:

1. A computer-implemented method of providing vehicle access, the method comprising:
receiving, from a user, a request to access a vehicle;
determining whether the user is one of a set of approved users;
determining a type of the request; and
granting the user a limited-access to the vehicle when:
the user is not one of the set of approved users; and
the type of the request is an emergency access request.

2. The computer-implemented method of claim 1 wherein the request is received from a mobile computing device.

3. The computer-implemented method of claim 2 further comprising:
receiving data corresponding to an identity of the user from the mobile computing device.

4. The computer-implemented method of claim 2 wherein the request further includes at least one of:
an access code assigned to the user, the access code associated with gaining user access to user-non-approved vehicles under an emergency condition;
an indication that an emergency button is activated on the mobile computing device; or
an indication that an emergency 9-1-1 call has been made within a threshold period of time by the mobile computing device.

5. The computer-implemented method of claim 1 wherein the request includes biometrics data corresponding to the user.

6. The computer-implemented method of claim 5 wherein the biometrics data includes at least one of:
fingerprint data;
iris data;
facial recognition data; or
DNA data.

7. The computer-implemented method of claim 5 further comprising:
sending the biometrics data to an authentication server; and
receiving confirmation that the biometrics data corresponds to the user,
wherein granting the user limited-access is further based on receiving the confirmation that the biometrics data corresponds to the user.

8. The computer-implemented method of claim 1 wherein determining the type of the request includes determining whether the request is at least one of a normal access request or an emergency access request.

9. The computer-implemented method of claim 1 wherein the limited-access to the vehicle includes initiating an advanced driver assistance system (ADAS) to automatically drive the user to one of a predetermined set of destinations.

10. The computer-implemented method of claim 9 wherein the limited-access to the vehicle prevents manual control of the vehicle by the user.

11. The computer-implemented method of claim 10 further comprising:
contacting an owner of the vehicle; and
informing the owner that the vehicle is in use by the user.

12. The computer-implemented method of claim 11 wherein informing the owner that the vehicle is in use by the user further includes at least one of:
providing an identity of the user; or
providing which of the predetermined set of destinations has been selected by the user.

13. The computer-implemented method of claim 9 further comprising:
receiving, from the user, a selection of one of the predetermined set of destinations;
contacting a person or entity associated with the selected destination of the predetermined set of destinations; and
providing user data to the contacted person or entity, the user data corresponding to the emergency access request.

14. The computer-implemented method of claim 13 wherein the user data corresponding to the emergency access request includes at least one of:
image data of the user, the image data generated by one or more image sensors in the vehicle; or
audio data of the user, the audio data generated by one or more microphones in the vehicle.

15. A system comprising:
one or more processors; and
one or more non-transitory computer-readable storage mediums containing instructions configured to cause the one or more processors to perform operations including:
receiving, from a user, a request to access a vehicle;
determining whether the user is one of a set of approved users;
determining a type of the request; and
granting the user a limited-access to the vehicle when:
the user is not one of the set of approved users; and
the type of the request is an emergency access request.

16. The system of claim 15 wherein the request further includes at least one of:
an access code assigned to the user, the access code associated with gaining user access to user-non-approved vehicles under an emergency condition;
an indication that an emergency button is activated on a mobile computing device; or
an indication that an emergency 9-1-1 call has been made within a threshold period of time by the mobile computing device.

17. The system of claim 15 further containing instructions configured to cause the one or more processors to perform operations including:
contacting an owner of the vehicle; and
informing the owner that the vehicle is in use by the user.

18. The system of claim 17 wherein informing the owner that the vehicle is in use by the user further includes at least one of:
providing an identity of the user; or
providing which of a predetermined set of destinations has been selected by the user.

19. A computer-implemented method of providing vehicle access, the method comprising:
means for receiving, from a user, a request to access a vehicle;
means for determining whether the user is one of a set of approved users;
means for determining a type of the request; and
means for granting the user a limited-access to the vehicle when:
the user is not one of the set of approved users; and
the type of the request is an emergency access request.
